(19) United States
(12) Patent Application Publication
(10) Pub. No.: US 2005/0123502 A1
(43) Pub. Date: Jun. 9, 2005

(54) Nicotine containing oral compositions

(76) Inventors: Shing Y. Chan, Parsippany, NJ (US); Stanley J. Lech, Parsippany, NJ (US); Kenneth R. Strahs, Parsippany, NJ (US)

Correspondence Address: GLAXOSMITHKLINE;Corporate Intellectual Property - UW2220, P.O. Box 1539, King of Prussia, PA, 19406-0939, US

(21) Appl. No.: 10/958,764

(22) Filed: Oct. 5, 2004

Related U.S. Application Data
(60) Provisional application No. 60/509,607, filed on Oct. 7, 2003.

(51) Int. Cl. A61K 31/785; A61K 31/715; A61K 31/4439

(52) U.S. Cl. 424/78.3; 514/54; 514/343

(57) ABSTRACT

An oral composition comprising a nicotine active, a polycarbophil component or a salt thereof, and an alginic acid component or a salt thereof, is provided. The oral composition provides high maximum plasma nicotine concentrations.

### BACKGROUND OF THE INVENTION

1. Field of the Invention

This invention relates to a certain oral dosage form comprising certain polymeric complexes and a nicotine active. The composition is suitable for achieving high maximum plasma concentrations when administered to a human.

2. Description of Related Art

Delivery of pharmaceutical compositions through the use of polymeric carriers is a well known technique. U.S. Pat. No. 4,615,697 to Robinson relates to the use of bioadhesive compositions for buccal administration. The buccal delivery systems disclosed in Robinson utilize known bioadhesives to hold the polymeric system in place in the buccal cavity after insertion therein. The drug is released from a bioadhesive matrix and absorbed into the buccal lining. The compositions disclosed in Robinson provide a means of trans-mucosal delivery of therapeutic agents that are subject to poor bioavailability due to solubility limitations, polarity considerations, degradation due to pH, enzymatic exposure or “first pass” metabolism by the liver or gastrointestinal enzymes after oral ingestion. However, such delivery systems, although of general utility have certain disadvantages in actual application.

U.S. Pat. No. 4,900,552 to Sanvordeker et al, provides a composition for releasing active ingredients in the buccal cavity itself for an extended period of time. The composition of this patent has a trilaminate film segment capable of delivering an active ingredient within the buccal cavity while attached to a wall of that cavity. The trilaminate film segment includes a hydratable bioadhesive base layer, a non-adhesive reservoir layer and a water-impermeable barrier sandwiched between and bonded to the base layer and the reservoir layer.

Alginic acid, including its salts, has also been used in various forms and combinations for purposes of providing bioadhesive compositions for the administration of active compositions. As one example thereof, the use of cross-linked alginate gum gel is described in U.S. Pat. No. 3,640,741 to Etes as being suitable for use as the bioadhesive.

U.S. Pat. No. 5,110,605 to Acharya generally discloses polymer complex compositions that comprise a reaction complex formed by the interaction of a polycarbophil component with aliginic acid or a salt thereof in the presence of a divalent cation and in the presence of an active ingredient.

In the present invention, it has been surprisingly discovered that certain compositions comprising a nicotine active, alginic acid, or salt thereof, and a polycarbophil component, or a salt thereof, provide higher maximum plasma concentrations of nicotine when administered to a human as compared to other oral dosage forms.

### SUMMARY OF THE INVENTION

The present invention provides an oral composition that comprises a nicotine active, alginic acid, or a salt thereof, and a polycarbophil component, or a salt thereof. A polymeric complex is formed through the interaction of the polycarbophil component, or a salt thereof, with alginic acid, or a salt thereof, in the presence of a divalent cation, as more particularly described in U.S. Pat. No. 5,110,605 to Archarya, incorporated herein by reference in its entirety. In one embodiment, the oral composition comprises nicotine polacrilex and a complex of calcium polycarbophil and sodium alginate. Because of the nature of the polymeric complexes of the present invention, the compositions of the present invention also exhibit some bioadhesive properties. Surprisingly, the compositions of the present invention achieves a higher maximum plasma level concentrations (Cₘₐₓ) when administered to a human as compared with other nicotine containing oral compositions.

### DETAILED DESCRIPTION OF THE INVENTION

The present invention relates to nicotine containing oral compositions that comprise a nicotine active and a reaction complex that is formed through the interaction of a polycarbophil component, or a salt thereof, with alginic acid, or a salt thereof, in the presence of a divalent cation. In one embodiment, the divalent cation is initially present in the form of a salt with the polycarbophil type composition, such as calcium polycarbophil. The compositions of the present invention surprisingly achieve higher maximum blood serum concentrations when administered to a human as compared to other nicotine containing oral compositions.

As indicated, due to the structure of the polymeric complexes of the present invention, the complexes may also be considered to be bioadhesives. Varying degrees of bioadhesive properties are exhibited by the complexes, as the alginic acid component is itself a bioadhesive. Calcium polycarbophil, which is the preferred form of the polycarbophil component, has no bioadhesive properties.

Nicotine Active

As used herein, the term “nicotine active” refers to one or more compounds selected from nicotine, derivatives of nicotine such as nicotine salts and nicotine complexes, tobacco extract or leaf, and other pharmacologically active compounds which are useful for reducing cravings for nicotine, such as lobeline. As used herein, “cravings for nicotine” include cravings associated with tobacco usage, such as smoking and chewing tobacco.

A variety of nicotine actives are well known in the art and are commercially available. Specific examples of nicotine actives suitable for use in the present invention include nicotine oil, nicotine bitartrate, and nicotine complexed with cyclodextrin or polymer resins, such as nicotine polacrilex. In one embodiment the nicotine active is nicotine polacrilex. The nicotine may be use in one or more distinct physical forms well know in the art, including free base forms, encapsulated forms, ionized forms, and spray-dried forms.

The oral dosage form comprises one or more nicotine actives in an amount effective to reduce nicotine cravings, preferably within one hour of starting oral administration. In general, the amount of nicotine may vary depending on the recommended or therapeutic dosage for the particular nicotine active. Such dosages are known or ascertainable by conventional methods by those of skill in the art. The composition typically contains from about 0.5 mg to about 5 mg of nicotine active per unit dosage form. In one embodiment, the composition contains from about 1 mg to about 4 mg of nicotine active per unit dosage form, and more preferably about 2 mg to about 4 mg of nicotine active per unit dosage form.

Excipients

One or more excipients may also be included with an active in the composition. In particular, one or more buffering materials are especially desirable to facilitate transmucosal absorption of nicotine actives. The buffer provides an alkaline mouth saliva pH that tends to enhance transmucosal absorption of such nicotine actives. Suitable buffer materials include inorganic or organic bases that have the capability to provide a mouth saliva pH of from above 7.0 to about 12.0. In one embodiment, the mouth saliva pH is from above 7.0 to about 11.0. In another embodiment, the mouth saliva pH is from about 7.5 to about 10.0. In yet another embodiment, the mouth saliva pH is from about 7.5 to about 9.0. Suitable buffer materials or agents include sodium carbonate, sodium bicarbonate, calcium carbonate, potassium carbonate, potassium bicarbonate, sodium phosphate dibasic, and sodium phosphate tribasic, potassium phosphate dibasic and potassium phosphate tribasic or any combinations thereof. In one embodiment, the buffer material comprises sodium carbonate and potassium bicarbonate.

Generally, a sufficient buffer is used such that the mouth saliva pH becomes and remains alkaline while the oral dosage form is held within the mouth during oral administration. When incorporate in the compositions of the present invention; the buffer generally is present from about 0.2% to about 5% of the total weight of the composition.

Chelating agents, such as ethylenediaminetetracetic acid (EDTA) that bind calcium ions and assist in passage of medicinal agents through the mucosa and into the blood stream are also suitable for use in the present compositions. Other chelating agents that may be used include, but are not limited to, calcium citrate, calcium disodium EDTA, B-cyclodextrin, diammonium EDTA, disodium EDTA, distarch phosphate, pentasodium pentatate, sodium citrate, potassium phosphate, tetraammonium EDTA; tetrahydroxypropyl ethylenediamine; tetrasodium EDTA, trisodium EDTA, trisodium HEDTA, D-gluconic acid, edetic acid, etidronic acid, and any combinations thereof. Another illustrative group of adjuvants are the quaternary nitrogen-containing compounds, such as benzalkonium chloride, that also assist medicinal agents in passing through the mucosa and into the blood stream.

In addition to the above ingredients, there may also be incorporated other excipients selected from among the various pharmaceutically acceptable excipients available to the those skilled in the art for the purpose of obtaining desirable processing and physical qualities including enhancement of the dispersibility and stability of the active ingredient. Such excipients include, but are not limited to, one or more fillers; binders; disintegrants; lubricants; thickeners; sweeteners; flavorants; emulsifiers/; antioxidants/preservatives; vitamins; tastemasking agents; and colorants.

The following binders that may be used include, but are not limited to starch, gelatin, chitin, chitosan, sucrose, glucose, dextrose, lactose, maltodextrin, acacia, guar gum, arabic, karaya, tragacanth, carrageenan, xanthan gum, lecithin, methylcellulose, ethylcellulose, cellulose ester, hydroxypropylcellulose, hydroxypropylmethylcellulose, hydroxypropylethylcellulose, sodium carboxymethylcellulose, microcrystalline cellulose (Avicels), polyvinylpyrrolidone, magnesium aluminum silicate, polyethylene glycol, polyvinyl acetate, PVP/VA copolymer, wax, albumin, water, alcohol, and any combinations thereof. The binder is preferably present in an amount of at least about 40%, in one embodiment at least about 50% and in another embodiment at least about 70% of the total weight of the composition.

Acceptable disintegrants that may be used in the present compositions include, but are not limited to, starch, such as sodium starch glycolate, potato starch, and corn starch; clay such as bentonite and kaolin; cellulose; carboxymethylcellulose; methylcellulose; crosslinked polyvinylpyrrolidone; guar gum; acacia; croscamellose of sodium; veegum; and any combinations thereof.

Examples of thickeners that may be used include, but are not limited to, polyethylene glycol, magnesium aluminum silicate, maltodextrin, D-mannitol, methylcellulose, microcrystalline cellulose, locust bean gum, hydroxyethylcellulose, hydroxypropylcellulose, hdroxypropylmethylcellulose, guar gum, karaya gum, isostearyl lactate, hectorite, bentonite, cetyl alcohol, poloxamine, polyethylene, polyethylene wax, polyvinyl acetate phthalate, polyvinylpyrrolidone, tragacanth gum, xanthan gum, sodium magnesium silicate, silica, sorbitan tristearate, calcium sodium, PVA/MA copolymer, carboxymethylcellulose, sodium carboxymethylcellulose, carrageenan, corn starch, and any combinations thereof.

The pharmaceutically acceptable lubricants in accordance with the present composition include, but are not limited to, polyethylene glycol, colloidal silicon dioxide, sodium stearyl fumarate, stearic acid and its pharmaceutically acceptable alkali metal salts (e.g. calcium stearate, sodium stearate), silica, sodium lauryl sulfate, sodium chloride, magnesium lauryl sulfate, vegetable oil, talc, and any combinations thereof. Lubricants are typically present at levels from about 0.5% to about 5.0% when incorporated in the compositions of the present invention.

Suitable fillers for use in the present composition include, but are not limited to, those conventionally used in the art, such as cellulose, monosaccharide e.g. lactose and glucose; disaccharide e.g. sucrose, polysaccharide e.g. mannitol; silicic acid, amaranth; dicalcium phosphate; tricalcium phosphate, microcrystalline cellulose; acacia gum; gum arabic; calcium sulfate; fructose; lactose; kaolin; mannitol; sorbitol; inositol; sodium chloride; maltodextrin; starch; powdered sugar; and any combinations thereof. When incorporated, fillers are generally present in the present compositions from about 50% to about 99%.

Suitable sweeteners include, but are not limited to, acesulfame potassium, acetophenone, acetyl butyryl, L-arabinose, aspartame, butyl lactate, calcium saccharin, corn syrup, dextrate, fructose, D-glactose, D-glycose (anhydride ormonohydrate), honey, isomalt, isophorone, lactitol, lactose, maltitol, maltose, D-mannitol, methyl salicylate, soluble saccharin salt (e.g. sodium, calcium salts), the free acid form of saccharin, cyclamate salt, the salts of 3,4-dihydro-6-methyl-1,2,3-oxathiazine-4-one-2,2-dioxide, xylitol, and any combinations thereof.

Flavorants that may be in the present composition include, but are not limited to, natural or synthetic flavors known in the art, such as mint (e.g. wintergreen, peppermint, spearmint), menthol, citrus (e.g. orange, lemon), other fruit flavors (e.g. cherry, grape), vanilla, cinnamon, tobacco, coffee, chocolate, nut derived flavors, and any combinations thereof.

Emulsifiers and surfactants that may be used in the present compositions include, but are not limited to, polyethylene glycol; poloxamer; pololamine; mono and diglycerides of fatty acids; glycerine, glyceride, polyvinyl alcohol; natural and synthetic gums, wax and oil; and the like, and any combinations thereof.

Antioxidant or preservative that may be used include, but are not limited to, benzoic acid and its pharmaceutically acceptable alkali metal salts, benzylparaben, BHT, BHA, salicylic acid, calcium ascorbate, sodium ascorbate, sodium borate, isobutylparaben, isopropylparaben, disodium EDTA, maleic acid, maleic anhydride, and the like, and any combinations thereof.

Vitamins for use in the present compositions include, but are not limited to, vitamins of the A group; the B group, such as thiamin, riboflavin, niacin, B-6, pantothenic acid, biotin, folacin and B-12; the D group; the E group; the K group; vitamin C; and any combinations thereof.

Tastemasking agents for use in the present composition include but are not limited to flavorants and sweeteners, such as high intensity sweeteners, methacrylic acid copolymer, microcrystalline wax, carnauba, beeswax, cellulose acetate phthalate, polyvinylacetate phthalate, PVA/MA copolymer, gelatin, and any combinations thereof.

Suitable colorants include pigment, dye and natural food color that are suitable for food and drug applications, e.g. FD&C dyes and lakes. Colorants typically comprise from about 0.001% to about 0.05% of the total weight of the composition when used.

Polycarbophil Component

Several types of materials are suitable for forming the polycarbophil type composition component. The polymer contains a plurality of a repeating unit of which at least about 80 percent contain at least one carboxyl functionality and about 0.05 to about 1.5 percent cross-linking agent substantially free from polyalkenyl polyether, with the percentages being based upon the weights of the unpolymerized repeating unit and cross-linking agent, respectively. In a more preferred embodiment, at least about 90 percent of the repeating units contain at least one carboxyl functionality. In a still more preferred embodiment, at least 95 percent of those repeating units contain at least one carboxyl functionality. Most preferably, this material is a reaction product of the polymerization of only a carboxyl-functional monomer and a cross-linking agent. Also in a more preferred embodiment, this component contains about 0.1 to about 1 percent by weight of polymerized cross-linking agent. This type of polymer is disclosed in U.S. Pat. No. 4,615, 697 to Robinson, which is incorporated herein by reference, and certain species of this type of polymer is commercially available under the generic name “polycarbophil”.

A polycarbophil type composition polymer component useful herein may thus be defined as a reaction product of the copolymerization of at least 80 weight percent monoethylenically unsaturated carboxy-functional monomer and about 0.05 to about 1.5 weight percent of a cross-linking agent free of polyalkenyl polyether. The remaining monomers that may be present to constitute 100 percent by weight of the monomers are discussed below.

In addition to the above two ingredients, the polycarbophil component may also include polymerized monoethylenically unsaturated repeating units such as C1-C6 alkyl esters of one or more of the above-described acids such as hexyl acrylate, butyl methacrylate and methyl crotonate; hydroxyalkylene-functional esters of the above-described acids that contain a per molecule average of 1 to about 4 oxyalkylene groups containing 2-3 carbon atoms such as hydroxyethyl methacrylate, hydroxypropyl acrylate and tetraethylene glycol monoacrylate; methacrylamide, acrylamide and their C1-C4 mon- and di-alkyl derivatives such as N-methyl acrylamide, N-butyl methacrylamide and N,N-dimethyl acrylamide; styrene; and the like as are known in the art as being copolymerizable with the above described carboxyl functionality-containing monomers and cross-linking agents. The polymers most preferably are prepared from only the monoethylenically unsaturated carboxy-functional monomer and the cross-linking agent.

The polycarbophil component useful herein may be prepared by conventional free radical polymerization techniques utilizing initiators, such as benzoyl peroxide, azobisisobutyronitrile, and the like, are polymerized in an aqueous medium, and are not agglomerated by steam action. A particularly preferred polyhydrophil component that is commercially available is that material sold under the designation calcium polycarbophil by the B. F. Goodrich Co. of Cleveland, Ohio. The United States Pharmacopeia, 1990. edition, United States Pharmacopeial Convention, Inc., Rockville; Md., at page 218, indicates that calcium polycarbophil is a calcium salt of polyacrylic acid cross-linked with divinyl glycol that has a calcium content of not less than 18% and not more than 22% and absorbs not less than 35 grams of sodium bicarbonate solution per one gram of the powder in the test for Absorbing power. Again, calcium polycarbophil, or any salts, especially alkali salts thereof, can be used in the present invention.

Alginic Acid

Alginic acid is a purified polysaccharide, linear polymer that is extracted from seaweed. It is available in many forms, especially as a calcium, potassium, or sodium salt, or as propylene glycol alginate. Preferably, sodium alginate is employed in the present composition or any alkali metal salts of any of these, or any combinations thereof.

Divalent Cation

The interaction of the polycarbophil type composition component with the alginic acid or alginate must occur in the presence of a divalent cation. Preferably, the divalent cation is calcium, although it may also be magnesium. In certain instances divalent iron, copper, or chromium may also be used. The divalent cation should be present in an amount from about 5 to about 25 percent and in one embodiment from about 18 to about 22 percent, based on the weight of polycarbophil. Most preferably, the calcium cation is originally present as the salt of the polycarbophil type composition. However, it may also be initially present as the salt of the alginic acid or may otherwise introduced as a calcium ion-containing compound, such as calcium chloride, calcium gluconate, calcium hydroxide, or the like.

The polycarbophil type component may be interacted with the alginic acid in any suitable means. Preferably, the alginic acid is present in the form of its sodium salt, while the polycarbophil type composition is present in the form of its calcium salt. Most preferably, the polycarbophil type composition component is that sold under the designation calcium polycarbophil.

Prior to interacting the two components, a nicotine active may be incorporated into one or both of the polycarbophil type composition and the alginate. The incorporation may be through dissolution, or dispersion of the active composition in a matrix of the alginate, for example, as a solid or a semi-solid or the alginate may be in an aqueous or mixed solvent system.

The alginate component may be employed in an amount from about 0.01% to about 25%, based on the total weight of the composition. In one embodiment, the alginate component is present in an amount about 0.1% to about 15% based on the total weight of the composition. In another embodiment, the alginate component is present from about 1% to about 10% by weight of the total composition. In yet another embodiment the alginate component is present in an amount from about 3% to about 6%. The amount of polycarbophil type composition will affect the consistency of the final product. Accordingly, the final product may vary from a water-like consistency to that of a solid dry powder. The polycarbohil component is generally present in an amount from about 0.01% to about 25% by weight of the total composition. In one embodiment the polycarbophil component is present from about 0.1% to about 15% by weight of the total composition. In another embodiment, the polycarbophil component is present from about 1% to about 5% by weight of the total composition. In yet another embodiment the polycarbophil composition is present from about 1% to about 3% by weight of the total composition. In one embodiment the total of alginate and polycarbophil is not more than about 25% of the total weight of the composition. In another embodiment the total of alginate and polycarbophil components is not more than 15% by weight of the composition. The polycarbophil component may be introduced into the reaction as a solid or in an aqueous or a mixed solvent system. All percentages expressed in this application are by weight unless stated otherwise.

The interaction of the two components in the presence of water or other polar solvent and in the presence of a bivalent cation, especially calcium, results in the formation of a complex that may be described as membrane type matrix, specifically at the interface of the two polymers. The resulting matrix structure then acts to control the diffusion or other transport of the active composition within and from the matrix itself.

The interaction of the polycarbophil type composition with the alginic acid or alginate should be at a pH of from about 3 to about 10, and in one embodiment at a pH from about 6.5 to about 7.5. Interaction at such a pH will assure that the polycarbophil and alginic acid components form the desired complex.

The rate or level of controlled or sustained release will vary, depending upon the ratio of the components employed, the particular active composition, the method of incorporation, the order of mixing of the components, and the like. Additional additives may also be present that may modify the characteristics of the matrix and its release properties.

Preferably, the nicotine active is incorporated into the alginate, prior to the interaction with the polycarbophil component.

In typical practice, the ratio by weight of the polymeric complex to the available nicotine active in the composition is about 100:1 to about 10:1. In preferred practice, however, the weight ratio of polymeric complex to the available nicotine active is about 50:1 to about 25:1. Those weight ratios are determined using dry ingredients and based on the amount of available nicotine active per unit dose form.

A composition of this invention may be provided in a variety of physical forms. For example, a composition may be a substantially uniformly mixed composition of the polymeric reaction complex and the nicotine active in either dry form, as a semi-solid or as a liquid suspension. That intimate mixture may, for example, be a mixture of dry solids, or of the active composition dissolved or suspended in a pharmaceutically or cosmetically acceptable (physiologically tolerable) carrier that also includes suspended particles of polymeric complex. In one embodiment, the compositions of the present invention are designed for oral administration in the form of a pill, a lozenge, candy, an orally dissolving film or similar product form, which retained in the oral cavity for a sufficient period to dissolved or disintegrate therein.

Illustrative of an intimate mixture of dry composition components is an admixed powder formed from comminuted polymeric complex particles having a size sufficient to pass through a 20 mesh sieve screen and be retained on a 200 mesh sieve screen (U.S. Standard Sieve Series) admixed with similarly or smaller sized particles of an active composition such as chlorothiazide. (Hereinafter, when particles are sized to pass through one screen and be retained on a second screen, as above, the size of the passing screen mesh will be written first, followed by a virgule, “/”, and then the size of the retaining screen mesh. Thus, the above passing and retaining screen mesh sizes are written “20/200”.) The mixture may be provided for treatment in tablet form or within a gelatin capsule and ingested for treatment.

The size of the polymeric complex-particles has some effect upon the compositions of this invention. It is apparent that the polymeric complex particles should not be so large that the composition cannot be administered without undue difficulty. For example, if the composition is to be swallowed, the polymeric complex particles must be sized to permit passage of the composition to the stomach without impeding passage of subsequently ingested foods or liquids thereto.

Typically, at the maximum, a useful polymeric complex is sized to pass through a sieve screen having a 10 mesh (U.S. Standard Sieve Series); i.e., a 2000 micron opening. Preferably, the polymeric complex particles are sized to pass through a 30 mesh sieve screen (U.S. Standard Sieve Series). Particles having a relatively small size swell more rapidly than do particles having a relatively large size, and thus, a relatively small size is preferred for the particles.

In another embodiment, the polymeric complex is swollen in an aqueous medium containing the active composition, and the active composition is sorbed (absorbed or adsorbed) into or onto the swollen polymeric complex particles. After drying, the composition so prepared is provided for treatment as described above. The word “dry” is used herein in relation to a polymeric complex to mean that the polymeric complex does not adhere when touched with a finger within a rubber glove, and is substantially unswollen.

The controlled release composition adheres to the skin or to mucus membranes (mucosa) in the presence of sufficient water to swell the polymeric complex.

### EXAMPLES

This example demonstrates the manufacture of a composition of the present invention in which benzocaine is incorporated for the purposes of achieving controlled release. Five-hundred milligrams of sodium alginate (Algin HV from Kelco) are mixed with eighty milligrams of benzocaine, USP. The resultant mass is granulated using 10% glycerol solution in purified water, USP, as the binding solution. Propylene glycol solution may also be employed in place of the glycerol. The resultant granulated mass is further mixed with five-hundred milligrams of calcium polycarbophil, USP. The mass is further granulated with the aid of purified water as a binder. Corn starch could also be employed at this granulation stage. An additional twenty milligrams of benzocaine are added to the mass with mixing. The resulting product is then conventionally formed into thin discs or wafers suitable for local administration.

The resulting discs are formulated of varying sizes, making them suitable for buccal, gingival or oral administration. The ratio of the polymeric complex material of the present invention to the weight of the active composition is approximately 10:1).

The following is exemplary of the compositions of the present invention:

| | Formula A1 | Formula A2 |
|---|---|---|
| Component | wt % | wt % |
| Mannitol | 86.26% | 85.45% |
| Sodium Alginate | 5.36% | 5.31% |
| Xanthan Gum | 1.03% | 1.02% |
| Potassium Bicarbonate | 0.24% | 0.23% |
| Calcium Polycarbophil | 2.67% | 2.64% |
| Sodium Carbonate | 1.91% | 1.90% |
| Nicotine Polacrilex (18% | 0.93% | 1.85% |
| w/w nicotine potency) | | |
| Magnesium Stearate | 1.00% | 1.00% |
| Aspartame | 0.50% | 0.50% |
| Mint Flavor | 0.10% | 0.10% |
| Total | 100.00% | 100.00% |

A comparison of a nicotine polacrilex containing lozenge for delivering 2 mg of nicotine active pursuant to Formula A1 above to another 2 mg nicotine polacrilex containing lozenge of Formula B ( a hard boiled lozenge formula).

| Formula B - Ingredients | wt % |
|---|---|
| Nicotine Polacrilex | 0.694 |
| Sodium Carbonate Anhydrous, NF | 0.770 |
| Isomalt (sugar alcohol mixture) | 97.684 |
| Acesulfame Potassium | 0.120 |
| Taste Masking | 0.588 |
| Anise Mint Flavor | 0.144 |

The following parameters were determined from the plasma concentration time data for nicotine.

| | |
|---|---|
| AUC(0-t): | Area under the plasma concentration versus time |
| | curve from time zero to time t, where t is the time of |
| | the last measurable plasma concentration of nicotine. |
| AUC(0-inf): | Area under the plasma concentration versus time |
| | curve from time zero extrapolated to infinity. |
| Cmax: | The highest observed plasma nicotine concentration. |
| Tmax: | Time to maximum plasma nicotine concentration |
| T½: | Half-life |

The objective of this study was to characterize the pharmacokinetic profile of Formula B and compare it with that of Formula A1. A single center, single dose, open-label, randomized, two-way crossover design was employed in which the subjects received 1 Formula B lozenge (Treatment B) and 1 Formula A1 (Treatment A1) lozenge under fasted conditions. 15 subjects completed the study. Five of the 15 subjects who completed the study were excluded from the analysis due to plasma nicotine concentrations at pre-dose greater than 5% of their respective Cmax values.

Cₘₐₓ for Treatment B was approximately 20% less than that of Treatment A1. The geometric mean ratio (90% Cl) for Cₘₐₓ comparing the Treatment B to Treatment A1 was 80.3% (67.2%, 96.0%). Time to maximal plasma nicotine concentration (Tₘₐₓ) was not significantly different between the two treatments (p=0.846); median Tₘₐₓ was 1.00 hr for Treatment B and 0.998 hr Treatment A1.

AUC₍₀₋ₜ₎ and AUC_{(0-inf)} for Treatment B were both about 16% less than that of Treatment A₁. The geometric mean ratio (90% Cl) for AUC₍₀₋ₜ₎ comparing Treatment B To Treatment A1 was 83.8% (72.1%, 97.4%). The geometric mean ratio (90% Cl) for AUC_{(0-inf)} comparing Treatment B to Treatment A1 was 83.6% (65.3%, 107.0%). These results indicate that the bioavailability of nicotine from the Treatment B was lower compared with the Treatment A1. It is concluded that this pilot study to compare pharmacokinetic profiles of Treatment B with Treatment A1 indicated that the present formulation of Treatment B was less bioavailable, as indicated by lower mean AUC₍₀₋ₜ₎ and AUC_{(0-inf)} values. The time to reach Cₘₐₓ (Tₘₐₓ) did not indicate a significant difference between the two formulations.

The arithmetic means of baseline adjusted plasma nicotine pharmacokinetic variables and the statistical comparisons of these variables following all three treatments are summarized in Table 1.

TABLE 1
Pharmacokinetic Variables of Baseline Adjusted Plasma
Nicotine for Treatment B vs. Treatment A1

| | Plasma Nicotine | | | | | |
|---|---|---|---|---|---|---|
| | Treatment B | | Treatment A1 | | | |
| | (n = 10) | | (n = 10) | | | |
| Pharmacokinetic | Arithmetic | | Arithmetic | | % Mean | 90% Cl* |
| Variable | Mean | SD | Mean | SD | Ratio* | (%) |
| AUC₍₀₋ₜ₎ (ng · hr/mL) | 13.14 | 9.200 | 15.38 | 8.365 | | |
| AUC_{(0-inf)} | 22.30 | 9.477 | 22.77 | 9.923 | | |
| (ng · hr/mL) | | | | | | |
| Cmax (ng/mL) | 4.700 | 1.915 | 5.836 | 1.816 | | |
| Tₘₐₓ (hr) | 0.971 | 0.331 | 0.919 | 0.287 | | |
| T_{1/2} (hr) | 1.86 | 0.354 | 1.76 | 0.397 | | |
| Kₑₗ (1/hr) | 0.385 | 0.0817 | 0.415 | 0.110 | | |
| In[AUC₍₀₋ₜ₎] | 2.352 | 0.7370 | 2.575 | 0.6429 | 83.8 | 72.1-97.4 |
| In[AUC_{(0-inf)}] | 3.042 | 0.3697 | 3.003 | 0.6160 | 83.6 | 65.3-107.0 |
| In(Cₘₐₓ) | 1.468 | 0.4312 | 1.716 | 0.3399 | 80.3 | 67.2-96.0 |

*= Based on analysis of In-transformed data

A study comparing the pharmacokinetic variations between a single 4 mg nicotine dose of Formula A2 (Treatment A2), Formula C (Treatment C) and Formula D (Treatment D) was conducted. Formulas C and D follow:

| Ingredient | Formula C | Formula D |
|---|---|---|
| Isomalt | 93.952 | 96.452 |
| Nicotine Polacrilex Resin | 0.962 | 0.962 |
| Sodium Carbonate | 0.770 | 0.770 |
| Xanthan Gum | 3.500 | 1.000 |
| Taste Masking Agent | 0.588 | 0.588 |
| Anise Mint Flavor | 0.144 | 0.144 |
| Acesulfame Potassium | 0.120 | 0.120 |

The parameters determined from the plasma concentration time data for nicotine were the same as those defined above. The arithmetic means of baseline adjusted plasma nicotine pharmacokinetic variables and the statistical comparisons of these variables following all three treatments are summarized in the Table 2 and Table 3.

TABLE 2
Pharmacokinetic Variables of Baseline Adjusted Plasma
Nicotine for Treatment C versus Treatment A2

| | Plasma Nicotine | | | | | |
|---|---|---|---|---|---|---|
| | Treatment C | | Treatment A2 | | | |
| | (n = 14) | | (n = 15) | | | |
| Pharmacokinetic | Arithmetic | | Arithmetic | | % Mean | |
| Variable | Mean | SD | Mean | SD | Ratio* | 90% Cl* (%) |
| AUC₍₀₋ₜ₎ (ng · hr/mL) | 32.20 | 33.61 | 35.89 | 32.33 | — | — |
| AUC_{(0-inf)} (ng · hr/mL) | 39.78 | 47.86 | 46.28 | 54.61 | — | — |
| Cₘₐₓ (ng/mL) | 8.746 | 4.729 | 9.997 | 3.301 | — | — |
| Tₘₐₓ (hr) | 1.32 | 0.667 | 1.15 | 0.652 | — | — |
| T_{1/2} (hr) | 2.35 | 1.24 | 2.44 | 1.64 | 96.2 | 83.6-108.8 |
| Kₑₗ (1/hr) | 0.356 | 0.143 | 0.347 | 0.128 | 103.2 | 94.0-112.4 |
| In[AUC₍₀₋ₜ₎] | 3.234 | 0.6066 | 3.393 | 0.5414 | 85.0 | 78.5-92.2 |
| In[AUC_{(0-inf)}] | 3.405 | 0.6249 | 3.566 | 0.6127 | 85.1 | 79.3-91.4 |
| In(Cₘₐₓ) | 2.080 | 0.4019 | 2.257 | 0.3097 | 84.2 | 77.6-91.4 |

*= Based on the LSMEAN values

Table 2 summarizes the results for the Treatment C versus Treatment A2. The mean ratios for AUC₍₀₋ₜ₎, AUC_{(0-inf)} and Cₘₐₓ were 85.0%, 85.1% and 84.2% respectively, indicating that Treatment C had a lower bioavailability than Treatment A2. Mean ratios for T_{1/2} and Kₑₗ were 96.2% and 103.2%, respectively, suggesting that Treatment C had similar elimination as Treatment A2. The 90% confidence intervals for the baseline adjusted AUC₍₀₋ₜ₎, AUC_{(0-inf)} and Cₘₐₓ for the comparisons of Treatment C and Treatment A2 were all outside the 80% to 125% range, and for baseline adjusted T_{1/2} and Kₑₗ for Treatment C and Treatment A2 were within the 80% to 125% range.

TABLE 3
Pharmacokinetic Variables of Baseline Adjusted Plasma
Nicotine for Treatment D versus Treament A2

| | Plasma Nicotine | | | | | |
|---|---|---|---|---|---|---|
| | Treatment D | | Treatment A2 | | | |
| | (n = 13) | | (n = 15) | | | |
| Pharmacokinetic | Arithmetic | | Arithmetic | | % Mean | 90% Cl* |
| Variable | Mean | SD | Mean | SD | Ratio* | (%) |
| AUC₍₀₋ₜ₎ (ng · hr/mL) | 31.55 | 31.99 | 35.89 | 32.33 | — | — |
| AUC_{(0-inf)} | 43.11 | 56.51 | 46.28 | 54.61 | — | — |
| (ng · hr/mL) | | | | | | |
| Cmax (ng/mL) | 8.346 | 3.446 | 9.997 | 3.301 | — | — |
| Tₘₐₓ (hr) | 1.25 | 0.674 | 1.15 | 0.652 | — | — |
| T_{1/2} (hr) | 2.51 | 1.85 | 2.44 | 1.64 | 101.2 | 88.3-114.1 |
| Kₑₗ (1/hr) | 0.357 | 0.149 | 0.347 | 0.128 | 102.5 | 93.1-111.9 |
| In[AUC₍₀₋ₜ₎] | 3.224 | 0.5886 | 3.393 | 0.5414 | 80.4 | 74.0-87.3 |
| In[AUC_{(0-inf)}] | 3.442 | 0.6630 | 3.566 | 0.6127 | 84.1 | 78.2-90.5 |
| In(Cₘₐₓ) | 2.068 | 0.3139 | 2.257 | 0.3097 | 81.3 | 74.8-88.4 |

*= Based on the LSMEAN values

Table 3 summarizes the results for Treatment D versus the Treatment A2. The mean ratios for AUC₍₀₋ₜ₎, AUC_{(0-inf)} and Cₘₐₓ were 80.4%, 84.1% and 81.3% respectively, indicating that Treatment D had a marginally lower bioavailability than Treatment A2. Mean ratios for T_{1/2}, and Kₑₗ were 101.2% and 102.5%, respectively, suggesting that Treatment D had similar elimination to Treatment A2. The 90% confidence intervals for the baseline adjusted AUC₍₀₋ₜ₎, AUC_{(0-inf)} and Cₘₐₓ, for the comparisons of Treatment D and Treatment A2 were outside the 80% to 125% range, and for baseline adjusted T_{1/2} and Kₑₗ were within the 80% to 125% range.

A third study was conducted to compare the pharmacokinetic variations between single, 2 mg nicotine dose according to Formula A1 (Treatment A1) and Formula E (Treatment E) and Formula F (Treatment F). Formulas E and F are follows:

| Ingredient | Formula E | Formula F |
|---|---|---|
| Nicotine polacrilex (NPA), USP* | 7.407 | 7.407 |
| Mannitol, USP (Pearlitol 200 SD) | 61.343 | 44.343 |
| Xanthan gum, NF/EP | 3.000 | 20.000 |
| Potassium bicarbonate, USP/EP | 0.250 | 0.250 |
| Sodium carbonate anhydrous, NF/EP | 2.000 | 2.000 |
| Sucralose, NF | 0.500 | 0.500 |
| Peppermint Oil H-7121(Nat&Art) | 2.000 | 2.000 |
| Natural Menthol-557620 APC0300 (Firmenich) | 20.000 | 20.000 |
| Mg stearate, NF/EP | 0.500 | 0.500 |
| Colloidal silicon dioxide, NF | 3.000 | 3.000 |

The parameters determined from the plasma concentration time data for nicotine were the same as those defined above.

TABLE 4
Pharmacokinetic Variables of Baseline Adjusted Plasma
Nicotine for Treatments E vs. Treatment A1

| | Plasma Nicotine | | | | | |
|---|---|---|---|---|---|---|
| | Treatment E | | Treatment A1 | | | |
| | (n = 10) | | (n = 13) | | | |
| Pharmacokinetic | Arithmetic | | Arithmetic | | % Mean | |
| Variable | Mean | SD | Mean | SD | Ratio* | 90% Cl* |
| Cₘₐₓ(ng/mL) | 3.670 | 1.115 | 4.520 | 1.934 | — | — |
| Tₘₐₓ(hr) | 0.651 | 0.269 | 0.809 | 0.522 | — | — |
| AUC₍₀₋ₜ₎(ng · hr/mL) | 11.02 | 6.919 | 14.85 | 7.985 | — | — |
| AUC_{(0-inf)}(ng · hr/mL) | 15.98 | 9.023 | 20.43 | 9.907 | — | — |
| T_{1/2}(hr) | 2.75 | 1.15 | 3.17 | 1.20 | 91.0 | 68.9-113.1 |
| Kₑₗ(1/hr) | 0.289 | 0.112 | 0.244 | 0.0767 | 114.6 | 92.9-136.3 |
| In(Cₘₐₓ) | 1.265 | 0.2736 | 1.429 | 0.4105 | 87.2 | 72.0-105.8 |
| In[AUC₍₀₋ₜ₎] | 2.283 | 0.4670 | 2.577 | 0.5009 | 79.0 | 68.1-91.7 |
| In[AUC_{(0-inf)}] | 2.673 | 0.4322 | 2.918 | 0.4561 | 82.7 | 72.9-93.8 |

Treatment A = 1 × Formula E
Treatment C = 1 × Formula A1
*= Based on the LSMEAN values

Table 4 summarizes the results for Treatment E vs. Treatment A1. The 90% confidence intervals for the baseline adjusted ln(Cₘₐₓ), ln[AUC₍₀₋ₜ₎], and In[AUC_{(0-inf)}] for the comparisons of 2 mg Treatment E and Treatment A1 were all outside the 80 to 125% range. Mean ratios for ln(Cₘₐₓ), ln[AUC₍₀₋ₜ₎], and ln[AUC_{(0-inf)}] were 87.2%, 79.0% and 82.7%, respectively, indicating that Treatment E had a lower bioavailability than Treatment A1. The 90% confidence intervals for baseline adjusted T_{1/2} and Kₑₗ were also outside the 80 to 125% range. Mean ratios were 91.0% and 114.6%, respectively, suggesting that Treatment E had a marginally faster elimination than Treatment A1.

TABLE 5
Pharmacokinetic Variables of Baseline Adjusted Plasma
Nicotine for Treatments F vs. Treatment A1

| | Plasma Nicotine | | | | | |
|---|---|---|---|---|---|---|
| | Treatment F | | Treatment A1 | | | |
| | (n = 12) | | (n = 13) | | | |
| Pharmacokinetic | Arithmetic | | Arithmetic | | % Mean | |
| Variable | Mean | SD | Mean | SD | Ratio* | 90% Cl* |
| Cmax(ng/mL) | 3.759 | 1.042 | 4.520 | 1.934 | — | — |
| Tₘₐₓ(hr) | 1.16 | 0.566 | 0.809 | 0.522 | — | — |
| AUC₍₀₋ₜ₎(ng · hr/mL) | 12.65 | 6.261 | 14.85 | 7.985 | — | — |
| AUC_{(0-inf)}(ng · hr/mL) | 19.12 | 8.387 | 20.43 | 9.907 | — | — |
| T_{1/2}(hr) | 3.53 | 1.71 | 3.17 | 1.20 | 104.8 | 85.0-124.7 |
| Kₑₗ(1/hr) | 0.244 | 0.113 | 0.244 | 0.0767 | 107.7 | 88.1-127.2 |
| In(Cₘₐₓ) | 1.285 | 0.2990 | 1.429 | 0.4105 | 87.5 | 73.3-104.6 |
| In[AUC₍₀₋ₜ₎] | 2.434 | 0.4696 | 2.577 | 0.5009 | 84.4 | 73.7-96.8 |
| In[AUC_{(0-inf)}] | 2.870 | 0.4112 | 2.918 | 0.4561 | 89.8 | 80.3-100.5 |

Treatment B = 1 × Formula F
Treatment C = 1 × Formula A1
*= Based on the LSMEAN values

Table 5 summarizes the results for Treatment F vs. Treatment A1. The 90% confidence intervals for the baseline adjusted In(Cₘₐₓ) and In [AUC₍₀₋ₜ₎] for the comparisons of Treatment F and Treatment A1 were outside the 80 to 125% range. The 90% confidence interval for the baseline adjusted AUC_{(0-inf)} for the comparisons of Treatment F and Treatment A1 was within the 80 to 125% range. Mean ratios for In(Cₘₐₓ), In[AUC₍₀₋ₜ₎], and In[AUC_{(0-inf)}] were 87.5%, 84.4%, and 89.8%, respectively, indicating that Treatment F had a marginally lower bioavailability than Treatment A1. The 90% confidence intervals for baseline adjusted T_{1/2} and Kₑₗ were within the 80 to 125% range. Mean ratios were 104.8% and 107.7%, respectively, suggesting that Treatment F had similar elimination as Treatment A1.

This invention has been described in terms of specific embodiments set forth in detail, but it should be understood that these are by way of illustration only and that the invention is not necessarily limited thereto. Modifications and variations will be apparent from this disclosure and may be resorted to without departing from the spirit of this invention, as those skilled in the art will readily understand. Accordingly, such variations and modifications of the disclosed products are considered to be within the purview and scope of this invention and the following claims.

1. An oral composition comprising:
a.) a nicotine active;
b.) a polycarbophil component; and
c.) an alginic acid component.

2. The oral composition of claim 1, wherein the polycarbophil component is calcium polycarbophil.

3. The oral composition of claim 2, wherein the alginic acid component is sodium alginate.

4. The oral composition of claim 1, wherein the nicotine active is nicotine polacrilex.

5. The oral composition of claim 1, wherein the amount of nicotine active is from about 0.5 mg to about 5 mg per unit dose.

6. The oral composition of claim 1, wherein the amount of nicotine active is from about 1 mg to about 4 mg per unit dose.

7. The oral composition of claim 1, wherein the amount of nicotine active is about 4 mg per unit dose.

8. The oral composition of claim 1, wherein the oral composition provides a maximum observed plasma concentration of nicotine of greater than about 9 ng/ml upon administration to a human.

9. The oral composition of claim 1, wherein the amount of nicotine active is about 2 mg per unit dose form.

10. The oral composition of claim 1, wherein the oral composition provides a maximum observed plasma concentration of nicotine greater than about 4 ng/ml upon administration to a human.

11. The oral composition of claim 1, further comprising a buffering agent.

12. The oral composition of claim 11, wherein the buffering agent comprises a mixture of sodium carbonate and potassium bicarbonate.

13. The oral composition of claim 11, wherein the buffering agent is present in an amount sufficient to maintain an alkaline mouth saliva pH while the oral dosage form is held within the mouth.

14. The oral composition of claim 1, wherein the nicotine active is selected from the group consisting of nicotine oil, nicotine bitartrate, nicotine complexed with cyclodextrin, nicotine complexed with polymer resin, and any combinations thereof.

15. The oral composition of claim 1, wherein the polycarbophil component is an alkali metal salt of calcium polycarbophil.

16. The oral composition of claim 1, wherein the polycarbophil component is present in an amount about 0.1% to about 90% of the total weight of the oral composition.

17. The oral composition of claim 1, wherein the polycarbophil component is present in an amount about 10% to about 15% of the total weight of the oral composition.

18. The oral composition of claim 1, wherein the alginic acid component is present in an amount about 0.1% to about 99.9% of the total weight of the oral composition.

19. The oral composition of claim 1, wherein the amount of nicotine active is about 2 mg to about 4 mg per unit dose.

20. The oral composition of claim 1, wherein the alginic acid component is an alkali metal salt of alginic acid.

21. The oral composition of claim 11, wherein the buffering agent is selected from the group consisting of sodium carbonate, sodium bicarbonate, calcium carbonate, potassium carbonate, potassium bicarbonate, sodium phosphate dibasic, sodium phosphate tribasic, potassium phosphate dibasic, potassium phosphate tribasic, and any combinations thereof.

22. The oral composition of claim 11, wherein the buffering agent is present in an amount about 0.2% to about 5% of the total weight of the oral composition.

23. The oral composition of claim 1, further comprising a chelating agent.

24. The oral composition of claim 23, wherein the chelating agent is selected from the group consisting of calcium citrate, calcium disodium EDTA, B-cyclodextrin, diammonium EDTA, disodium EDTA, distarch phosphate, pentasodium pentatate, sodium citrate, potassium phosphate, tetraammonium EDTA, tetrahydroxypropyl ethylenediamine, tetrasodium EDTA, trisodium EDTA, trisodium HEDTA, D-gluconic acid, edetic acid, etidronic acid, and any combinations thereof.

25. The oral composition of claim 1, further comprising a binder.

26. The oral composition of claim 25, wherein the binder is selected from the group consisting of starch, gelatin, chitin, chitosan, sucrose, glucose, dextrose, lactose, maltodextrin, acacia, guar gum, arabic, karaya, tragacanth, carrageenan, gum, xanthan gum, lecithin, methylcellulose, ethylcellulose, cellulose esters, hydroxypropylcellulose, hydroxypropylmethylcellulose, hydroxypropylethylcellulose, sodium carboxymethylcellulose, microcrystalline cellulose, polyvinylpyrrolidone, magnesium aluminum silicate, polyethylene glycol, polyvinyl acetate, PVP/VA copolymer, wax, albumin, water, alcohol, and any combinations thereof.

27. The oral composition of claim 1, further comprising a disintegrant.

28. The oral composition of claim 27, wherein the disintegrant is selected from the group consisting of sodium starch glycolate, potato starch, corn starch, bentonite, kaolin, cellulose, carboxymethylcellulose, methylcellulose, crosslinked polyvinylpyrrolidone, guar gum, acacia, crosscamellose of sodium, veegum, and any combinations thereof.

29. The oral composition of claim 1, further comprising a thickener.

30. The oral composition of claim 29, wherein the thickener is selected from the group consisting of polyethylene glycol, magnesium aluminum silicate, maltodextrin, D-mannitol, methylcellulose, microcrystalline cellulose, locust bean gum, hydroxyethylcellulose, hydroxypropylcellulose, hdroxypropylmethylcellulose, guar gum, karaya gum, isostearyl lactate, hectorite, bentonite, cetyl alcohol, poloxamine, polyethylene, polyethylene wax, polyvinyl acetate phthalate, polyvinylpyrroidone, tragacanth gum, xanthan gum, sodium magnesium silicate, silica, sorbitan tristearate, calcium sodium PVA/MA copolymer, carboxymethylcellulose, sodium carboxymethylcellulose, carrageenan, corn starch, and any combinations thereof.

31. The oral composition of claim 1, further comprising a filler.

32. The oral composition of claim 31, wherein the filler is selected from the group consisting of cellulose, monosaccharide, disaccharide, polysaccharide, mannitol; silicic acid, amaranth, dicalcium phosphate, tricalcium phosphate, microcrystalline cellulose, acacia gum, gum arabic, calcium sulfate, fructose, lactose, kaolin, mannitol, sorbitol, inositol, sodium chloride, maltodextrin, starch, powdered sugar, and any combinations thereof.

33. The oral composition of claim 32, wherein the filler is present in an amount about 50% and about 99% of the total weight of the oral composition.

34. The oral composition of claim 1, further comprising a lubricant.

35. The oral composition of claim 34, wherein the lubricant is selected from the group consisting of polyethylene glycol, colloidal silicon dioxide, sodium stearyl fumarate, magnesium stearate, stearic acid, pharmaceutically acceptable alkali metal salts of stearate acid, silica, sodium lauryl sulfate, sodium chloride, magnesium lauryl sulfate, vegetable oils, talc, and any combinations thereof.

36. The oral composition of claim 34, wherein the lubricant is present in an amount about 0.5% and about 5% of the total weight of the oral composition.

37. The oral composition of claim 1, further comprising a sweetener.

38. The oral composition of claim 37, wherein the sweetener is selected from the group consisting of acesulfame potassium, acetophenone, acetyl butyryl, L-arabinose, aspartame, butyl lactate, calcium saccharin, corn syrup, dextrate, fructose, D-glactose, D-glycose, honey, isomalt, isophorone, lactitol, lactose, maltitol, maltose, D-mannitol, methyl salicylate, soluble saccharin salt, the free acid form of saccharin, cyclamate salt, the salts of 3,4-dihydro-6-methyl-1,2,3-oxathiazine-4-one-2,2-dioxide, sylitol, and any combinations thereof.

39. The oral composition of claim 1, further comprising a flavorant.

40. The oral composition of claim 39, wherein the flavorant is selected from the group consisting of mint flavor, menthol, fruit flavor, vanilla, cinnamon, tobacco, coffee, chocolate, nut flavor, and any combinations thereof.

41. The oral composition of claim 1, further comprising an emulsifier.

42. The oral composition of claim 41, wherein the emulsifier is selected from the group consisting of polyethylene glycol, poloxamer, pololamine, monoglyceride, diglycerides of fatty acids, glycerine, glyceride, polyvinyl alcohol, natural gum, synthetic gum, wax, oil, and any combinations thereof.

43. The oral composition of claim 1, further comprising a preservative.

44. The oral composition of claim 43, wherein the preservative is selected from the group consisting of benzoic acid, pharmaceutically acceptable alkali metal salts of benzoic acid, benzylparaben, BHT, BHA, salicylic acid, calcium ascorbate, sodium ascorbate, sodium borate, isobutylparaben, isopropylparaben, disodium EDTA, maleic acid, maleic anhydride, and any combinations thereof.

45. The oral composition of claim 1, further comprising a vitamin.

46. The oral composition of claim 45, wherein the vitamin is selected from the group consisting of vitamins of the A group, vitamins of the B group, vitamins of the D group, vitamins of the E group, vitamins of the K group, vitamin C, and any combinations thereof.

47. The oral composition of claim 1, further comprising a tastemasking agent.

48. The oral composition of claim 49, wherein the tastemasking agent is selected from the group consisting of sweetener, flavorant, methacrylic acid copolymer, microcrystalline wax, carnauba, beeswax, cellulose acetate phthalate, polyvinylacetate phthalate, PVA/MA copolymer, gelatin, and any combinations thereof.

49. The oral composition of claim 1, further comprising a colorant.

50. The oral composition of claim 49, wherein the colorant is selected from the group consisting of pigment, dye, natural food color, and any combinations thereof.

51. The oral composition of claim 49, wherein the colorant is present in an amount about 0.0001% and about 0.05% of the total weight of the oral composition.

52. An oral dosage form comprising from about 3 mg to about 5 mg of a nicotine active wherein the dosage form provides a maximum observed plasma concentration of nicotine greater than about 9 ng/ml upon administration to a human.

53. The oral dosage form of claim 52, further comprising a polycarbophil component.

54. The oral dosage form of claim 53, wherein the polycarbophil component is calcium polycarbophil.

55. The oral composition of claim 52, wherein the polycarbophil component is an alkali metal salt of calcium polycarbophil.

56. The oral composition of claim 52, wherein the polycarbophil component is present in an amount about 0.1% to about 90% of the total weight of the oral composition.

57. The oral composition of claim 52, wherein the polycarbophil component is present in an amount about 10% to about 15% of the total weight of the oral composition.

58. The oral dosage form of claim 52, further comprising an alginic acid component.

59. The oral dosage form of claim 58, wherein the alginic acid component is sodium alginate.

60. The oral composition of claim 58, wherein the alginic acid component is present in an amount about 0.1% to about 99.9% of the total weight of the oral composition.

61. The oral composition of claim 58, wherein the alginic acid component is an alkali metal salt of alginic acid.

62. The oral dosage form of claim 52, further comprising at least one buffering agent.

63. The oral dosage form of claim 62, wherein the buffering agent comprises a mixture of sodium carbonate and potassium bicarbonate.

64. The oral composition of claim 62, wherein the buffering agent is selected from the group consisting of sodium carbonate, sodium bicarbonate, calcium carbonate, potassium carbonate, potassium bicarbonate, sodium phosphate dibasic, sodium phosphate tribasic, potassium phosphate dibasic, potassium phosphate tribasic, and any combinations thereof.

65. The oral composition of claim 62, wherein the buffering agent is present in an amount about 0.2% to about 5% of the total weight of the oral composition.

66. The oral composition of claim 52, wherein the nicotine active is selected from the group consisting of nicotine oil, nicotine bitartrate, nicotine complexed with cyclodextrin, nicotine complexed with polymer resin, and any combinations thereof.

67. The oral composition of claim 52, further comprising a chelating agent.

68. The oral composition of claim 67, wherein the chelating agent is selected from the group consisting of calcium citrate, calcium disodium EDTA, B-cyclodextrin, diammonium EDTA, disodium EDTA, distarch phosphate, pentasodium pentatate, sodium citrate, potassium phosphate, tetraammonium EDTA, tetrahydroxypropyl ethylenediamine, tetrasodium EDTA, trisodium EDTA, trisodium HEDTA, D-gluconic acid, edetic acid, etidronic acid, and any combinations thereof.

69. The oral composition of claim 52, further comprising a binder.

70. The oral composition of claim 69, wherein the binder is selected from the group consisting of starch, gelatin, chitin, chitosan, sucrose, glucose, dextrose, lactose, maltodextrin, acacia, guar gum, arabic, karaya, tragacanth, carrageenan, xanthan gum, lecithin, methylcellulose, ethylcellulose, cellulose ester, hydroxypropylcellulose, hydroxypropylmethylcellulose, hydroxypropylethylcellulose, sodium carboxymethylcellulose, microcrystalline cellulose, polyvinylpyrrolidone, magnesium aluminum silicate, polyethylene glycol, polyvinyl acetate, PVP/VA copolymer, wax, albumin, water, alcohol, and any combinations thereof.

71. The oral composition of claim 52, further comprising a disintegrant.

72. The oral composition of claim 71, wherein the disintegrant is selected from the group consisting of sodium starch glycolate, potato starch, corn starch, bentonite, kaolin, cellulose, carboxymethylcellulose, methylcellulose, crosslinked polyvinylpyrrolidone, guar gum, acacia, crosscamellose of sodium, veegum, and any combinations thereof.

73. The oral composition of claim 52, further comprising a thickener.

74. The oral composition of claim 73, wherein the thickener is selected from the group consisting of polyethylene glycol, magnesium aluminum silicate, maltodextrin, D-mannitol, methylcellulose, microcrystalline cellulose, locust bean gum, hydroxyethylcellulose, hydroxypropylcellulose, hdroxypropylmethylcellulose, guar gum, karaya gum, isostearyl lactate, hectorite, bentonite, cetyl alcohol, poloxamine, polyethylene, polyethylene wax, polyvinyl acetate phthalate, polyvinylpyrroidone, tragacanth gum, xanthan gum, sodium magnesium silicate, silica, sorbitan tristearate, calcium sodium PVA/MA copolymer, carboxymethylcellulose, sodium carboxymethylcellulose, carrageenan, corn starch, and any combinations thereof.

75. The oral composition of claim 52, further comprising a filler.

76. The oral composition of claim 75, wherein the filler is selected from the group consisting of cellulose, monosaccharide, disaccharide; polysaccharide, mannitol, silicic acid, amaranth, dicalcium phosphate, tricalcium phosphate, microcrystalline cellulose, acacia gum, gum arabic, calcium sulfate, fructose, lactose, kaolin, mannitol, sorbitol, inositol, sodium chloride, maltodextrin, starch, powdered sugar, and any combinations thereof.

77. The oral composition of claim 75, wherein the filler is present in an amount about 50% and about 99% of the total weight of the oral composition.

78. The oral composition of claim 52, further comprising a lubricant.

79. The oral composition of claim 78, wherein the lubricant is selected from the group consisting of polyethylene glycol, colloidal silicon dioxide, sodium stearyl fumarate, magnesium stearate, stearic acid, pharmaceutically acceptable alkali metal salts of stearic acid, silica, sodium lauryl sulfate, sodium chloride, magnesium lauryl sulfate, vegetable oil, talc, and any combinations thereof.

80. The oral composition of claim 78, wherein the lubricant is present in an amount about 0.5% and about 5% of the total weight of the oral composition.

81. The oral composition of claim 52, further comprising a sweetener.

82. The oral composition of claim 81, wherein the sweetener is selected from the group consisting of acesulfame potassium, acetophenone, acetyl butyryl, L-arabinose, aspartame, butyl lactate, calcium saccharin, corn syrup, dextrate, fructose, D-glactose, D-glycose, honey, isomalt, isophorone, lactitol, lactose, maltitol, maltose, D-mannitol, methyl salicylate, soluble saccharin salt, the free acid form of saccharin, cyclamate salt, the salts of 3,4-dihydro-6-methyl-1,2,3-oxathiazine-4-one-2,2-dioxide, sylitol, and any combinations thereof.

83. The oral composition of claim 52, further comprising a flavorant.

84. The oral composition of claim 83, wherein the flavorant is selected from the group consisting of mint flavor, menthol, fruit flavor, vanilla, cinnamon, tobacco, coffee, chocolate, nut flavor, and any combinations thereof.

85. The oral composition of claim 52, further comprising an emulsifier.

86. The oral composition of claim 85, wherein the emulsifier is selected from the group consisting of polyethylene glycol, poloxamer, pololamine, monoglyceride, diglycerides of fatty acids, glycerine, glyceride, polyvinyl alcohol, natural gum, synthetic gum, wax, oil, and any combinations thereof.

87. The oral composition of claim 52, further comprising a preservative.

88. The oral composition of claim 87, wherein the preservative is selected from the group consisting of benzoic acid, pharmaceutically acceptable alkali metal salts of benzoic acid, benzylparaben, BHT, BHA, salicylic acid, calcium ascorbate, sodium ascorbate, sodium borate, isobutylparaben, isopropylparaben, disodium EDTA, maleic acid, maleic anhydride, and any combinations thereof.

89. The oral composition of claim 52, further comprising a vitamin.

90. The oral composition of claim 89, wherein the vitamin is selected from the group consisting of vitamins of the A group, vitamins of the B group, vitamins of the D group, vitamins of the E group; vitamins of the K group; vitamin C, and any combinations thereof.

91. The oral composition of claim 52, further comprising a tastemasking agent.

92. The oral composition of claim 91, wherein the tastemasking agent is selected from the group consisting of sweetener, flavorant, methacrylic acid copolymer, microcrystalline wax, carnauba, beeswax, cellulose acetate phthalate, polyvinylacetate phthalate, PVA/MA copolymer, gelatin, and any combinations thereof.

93. The oral composition of claim 52, further comprising a colorant.

94. The oral composition of claim 93, wherein the colorant is selected from the group consisting of pigment, dye, natural food color, and any combinations thereof.

95. The oral composition of claim 93, wherein the colorant is present in an amount about 0.0001% and about 0.05% of the total weight of the oral composition.

96. An oral dosage form comprising from about 1 mg to about 3 mg of a nicotine active wherein the dosage form provides a maximum observed plasma concentration of nicotine greater than about 4 ng/ml upon administration to a human.

97. The oral dosage form of claim 96, wherein the maximum observed plasma concentration is greater than about 4.5 ng/ml upon administration to a human.

98. The oral dosage form of claim 96, further comprising a polycarbophil component.

99. The oral dosage form of claim 98, wherein the polycarbophil component is calcium polycarbophil.

100. The oral composition of claim 98, wherein the polycarbophil component is an alkali metal salt of calcium polycarbophil.

101. The oral composition of claim 98, wherein the polycarbophil component is present in an amount about 0.1% to about 90% of the total weight of the oral composition.

102. The oral composition of claim 98, wherein the polycarbophil component is present in an amount about 10% to about 15% of the total weight of the oral composition.

103. The oral dosage form of claim 96, further comprising an alginic acid component.

104. The oral dosage form of claim 103, wherein the alginic acid component is sodium alginate.

105. The oral composition of claim 103, wherein the alginic acid component is present in an amount about 0.1% to about 99.9% of the total weight of the oral composition.

106. The oral composition of claim 103, wherein the alginic acid component is an alkali metal salt of alginic acid.

107. The oral dosage form of claim 96, further comprising at least one buffering agent.

108. The oral dosage form of claim 107, wherein the buffering agent comprises a mixture of sodium carbonate and potassium bicarbonate.

109. The oral composition of claim 107, wherein the buffering agent is selected from the group consisting of sodium carbonate, sodium bicarbonate, calcium carbonate, potassium carbonate, potassium bicarbonate, sodium phosphate dibasic, sodium phosphate tribasic, potassium phosphate dibasic, potassium phosphate tribasic, and any combinations thereof.

110. The oral composition of claim 107, wherein the buffering agent is present in an amount about 0.2% to about 5% of the total weight of the oral composition.

111. The oral composition of claim 96, wherein the nicotine active is selected from the group consisting of nicotine oil, nicotine bitartrate, nicotine complexed with cyclodextrin, nicotine complexed with polymer resin, and any combinations thereof.

112. The oral composition of claim 96, further comprising a chelating agent.

113. The oral composition of claim 112, wherein the chelating agent is selected from the group consisting of calcium citrate, calcium disodium EDTA, B-cyclodextrin, diammonium EDTA, disodium EDTA, distarch phosphate, pentasodium pentatate, sodium citrate, potassium phosphate, tetraammonium EDTA; tetrahydroxypropyl ethylenediamine, tetrasodium EDTA, trisodium EDTA, trisodium HEDTA, D-gluconic acid, edetic acid, etidronic acid, and any combinations thereof.

114. The oral composition of claim 96, further comprising a binder.

115. The oral composition of claim 114, wherein the binder is selected from the group consisting of starch, gelatin, chitin, chitosan, sucrose, glucose, dextrose, lactose, maltodextrin, acacia, guar gum, arabic, karaya, tragacanth, carrageenan, xanthan gum, lecithin, methylcellulose, ethylcellulose, cellulose ester, hydroxypropylcellulose, hydroxypropylmethylcellulose, hydroxypropylethylcellulose, sodium carboxymethylcellulose, microcrystalline cellulose, polyvinylpyrrolidone, magnesium aluminum silicate, polyethylene glycol, polyvinyl acetate, PVP/VA copolymer, wax, albumin, water, alcohol, and any combinations thereof.

116. The oral composition of claim 96, further comprising a disintegrant.

117. The oral composition of claim 116, wherein the disintegrant is selected from the group consisting of sodium starch glycolate, potato starch, corn starch, bentonite, kaolin, cellulose, carboxymethylcellulose, methylcellulose, crosslinked polyvinylpyrrolidone, guar gum, acacia, croscamellose of sodium, veegum, and any combinations thereof.

118. The oral composition of claim 96, further comprising a thickener.

119. The oral composition of claim 118, wherein the thickener is selected from the group consisting of polyethylene glycol, magnesium aluminum silicate, maltodextrin, D-mannitol, methylcellulose, microcrystalline cellulose, locust bean gum, hydroxyethylcellulose, hydroxypropylcellulose, hdroxypropylmethylcellulose, guar gum, karaya gum, isostearyl lactate, hectorite, bentonite, cetyl alcohol, poloxamine, polyethylene, polyethylene wax, polyvinyl acetate phthalate, polyvinylpyrroidone, tragacanth gum, xanthan gum, sodium magnesium silicate, silica, sorbitan tristearate, calcium sodium PVA/MA copolymer, carboxymethylcellulose, sodium carboxymethylcellulose, carrageenan, corn starch, and any combinations thereof.

120. The oral composition of claim 96, further comprising a filler.

121. The oral composition of claim 120, wherein the filler is selected from the group consisting of cellulose, monosaccharide, disaccharide, polysaccharide, mannitol, silicic acid, amaranth, dicalcium phosphate, tricalcium phosphate, microcrystalline cellulose, acacia gum, gum arabic, calcium sulfate, fructose, lactose, kaolin, mannitol, sorbitol, inositol, sodium chloride, maltodextrin, starch, powdered sugar, and any combinations thereof.

122. The oral composition of claim 121, wherein the filler is present in an amount about 50% and about 99% of the total weight of the oral composition.

123. The oral composition of claim 96, further comprising a lubricant.

124. The oral composition of claim 123, wherein the lubricant is selected from the group consisting of polyethylene glycol, colloidal silicon dioxide, sodium stearyl fumarate, magnesium stearate, stearic acid, pharmaceutically acceptable alkali metal salts of stearic acid, silica, sodium lauryl sulfate, sodium chloride, magnesium lauryl sulfate, vegetable oil, talc, and any combinations thereof.

125. The oral composition of claim 123, wherein the lubricant is present in an amount about 0.5% and about 5% of the total weight of the oral composition.

126. The oral composition of claim 96, further comprising a sweetener.

127. The oral composition of claim 126, wherein the sweetener is selected from the group consisting of acesulfame potassium, acetophenone, acetyl butyryl, L-arabinose, aspartame, butyl lactate, calcium saccharin, corn syrup, dextrate, fructose, D-glactose, D-glycose, honey, isomalt, isophorone, lactitol, lactose, maltitol, maltose, D-mannitol, methyl salicylate, soluble saccharin salt, the free acid form of saccharin, cyclamate salt, the salts of 3,4-dihydro-6-methyl-1,2,3-oxathiazine-4-one-2,2-dioxide, sylitol, and any combinations thereof.

128. The oral composition of claim 96, further comprising a flavorant.

129. The oral composition of claim 128, wherein the flavorant is selected from the group consisting of mint flavor, menthol, fruit flavor, vanilla, cinnamon, tobacco, coffee, chocolate, nut flavor, and any combinations thereof.

130. The oral composition of claim 96, further comprising an emulsifier.

131. The oral composition of claim 130, wherein the emulsifier is selected from the group consisting of polyethylene glycol, poloxamer, pololamine, monoglyceride, diglycerides of fatty acids, glycerine, glyceride, polyvinyl alcohol; natural gum, synthetic gum, wax, oil, and any combinations thereof.

132. The oral composition of claim 96, further comprising a preservative.

133. The oral composition of claim 132, wherein the preservative is selected from the group consisting of benzoic acid, pharmaceutically acceptable alkali metal salts of benzoic acid, benzylparaben, BHT, BHA, salicylic acid, calcium ascorbate, sodium ascorbate, sodium borate, isobutylparaben, isopropylparaben, disodium EDTA, maleic acid, maleic anhydride, and any combinations thereof.

134. The oral composition of claim 96, further comprising a vitamin.

135. The oral composition of claim 134, wherein the vitamin is selected from the group consisting of vitamins of the A group, vitamins of the B group, vitamins of the D group, vitamins of the E group, vitamins of the K group; vitamin C, and any combinations thereof.

136. The oral composition of claim 96, further comprising a tastemasking agent.

137. The oral composition of claim 136, wherein the tastemasking agent is selected from the group consisting of sweetener, flavorant, methacrylic acid copolymers, microcrystalline wax, carnauba, beeswax, cellulose acetate phthalate, polyvinylacetate phthalate, PVA/MA copolymer, gelatin, and any combinations thereof.

138. The oral composition of claim 96, further comprising a colorant.

139. The oral composition of claim 138, wherein the colorant is selected from the group consisting of pigment, dye, natural food color, and any combinations thereof.

140. The oral composition of claim 138, wherein the colorant is present in an amount about 0.0001% and about 0.05% of the total weight of the oral composition.

141. An oral dosage form comprising a nicotine active and a polymer complex formed from the interaction of a polycarbophil component and an alginic acid component wherein the ratio of weight percent of polymer complex to weight percent of nicotine active present in the dosage form is from about 100:1 to about 10:1

142. The oral dosage form of claim 141, wherein the ratio of weight percent of polymer complex to weight percent of nicotine active present in the dosage form is from about 50:1 to about 25:1

143. The oral dosage form of claim 142, wherein the nicotine active is present from about 3 mg to about 5 mg per unit dose.

144. The oral dosage form of claim 143, wherein the maximum observed plasma concentration is greater than about 9 ng/ml upon administration to a human.
